(19) United States
(12) Patent Application Publication
(10) Pub. No.: US 2003/0108552 A1
(43) Pub. Date: Jun. 12, 2003

(54) Human inhibitor of apoptosis gene 1

(76) Inventors: Wei-Wu He, Columbia, MD (US); Peter L. Hudson, Germantown, MD (US); Craig A. Rosen, Laytonsville, MD (US)

Correspondence Address: HUMAN GENOME SCIENCES INC, 9410 KEY WEST AVENUE, ROCKVILLE, MD, 20850

(21) Appl. No.: 10/323,643

(22) Filed: Dec. 20, 2002

Related U.S. Application Data
(62) Division of application No. 08/464,588, filed on Jun. 5, 1995.
Application No. 08/464,588 is a continuation-in-part of application No. PCT/US95/05922, filed on May 11, 1995.

(51) Int. Cl. A61K 39/395; G01N 33/53; G01N 33/537; G01N 33/543; C12N 5/06; C07K 16/40

(52) U.S. Cl. 424/146.1; 530/388.26; 435/7.92; 435/338

(57) ABSTRACT

A human inhibitor of apoptosis polypeptide and DNA (RNA) encoding such polypeptide and a procedure for producing such polypeptide by recombinant techniques is disclosed. Also disclosed are methods for utilizing such polypeptide for the treatment of degenerative diseases, rheumatoid arthritis, septic shock, as an antiviral defense mechanism and to prevent the death of cells during trauma and strokes. Antagonists against such polypeptides and their use as a therapeutic to promote cell development, kill viral infections, promote tissue differentiation and development and maintain tissue homeostasis are also disclosed (tumors). Diagnostic methods for detecting mutations in the nucleic acid sequence encoding hIAP-1 protein are also disclosed.

This application is a divisional of, and claims the benefit of priority under 35 U.S.C. §120 of U.S. application Ser. No. 08/464,588, filed Jun. 5, 1995 which is a continuation-in-part of and claims the benefit of priority under 35 U.S.C. §120 of copending International Patent Application No. PCT/US95/05922, filed May 11, 1995.

This invention relates to newly identified polynucleotides, polypeptides encoded by such polynucleotides, the use of such polynucleotides and polypeptides, as well as the production of such polynucleotides and polypeptides. More particularly, the polypeptide of the present invention is human inhibitor of apoptosis gene 1, sometimes hereinafter referred to as “hIAP-1”. The invention also relates to inhibiting the action of such polypeptides.

Programmed cell death (apoptosis) is a process through which organisms get rid of unwanted cells. Programmed cell death can be considered as a specific type of terminal cell differentiation. During apoptosis, the cells of an organism round up and they have active blebbing at the cell surface, forming apoptotic bodies, the nuclear membranes and some internal structures break down, the nuclear DNA is fragmented by enzymes, and finally the cell breaks into pieces.

The early studies of apoptosis provided that drugs that block protein synthesis prevent apoptosis, suggesting that programmed cell death requires specific proteins. A key aid in finding the gene encoding the protein which is responsible for apoptosis was the emergence in the 1980's of the tiny, transparent round worm Caenorhabditis elegans as a valuable resource for identifying genes active in embryonic development. This microscopic worm has only 1,090 cells, and development biologists, therefore, were able to trace the lineage of each cell as the worm matures, and found that 131 of the embryonic cells undergo programmed cell death. Among the genes identified in C. elegans where the death genes ced-3 and ced-4, as well as ced-9, an “anti-death” gene which protects cells from apoptosis.

The search for mammalian analogs to these genes had a breakthrough with the discovery that the gene bcl-2, which had been identified as a cancer-causing oncogene, protects immune cells call lymphocytes from suicide and protects neurons as well. The worm protection gene, ced-9, was found to be 23% homologous to bcl-2, and the bcl-2 gene could substitute for ced-9 in C. elegans, rescuing ced-9 mutants from cell death.

Further, a match was found between the ced-3 and a new mammalian gene which coded for interleukin- -converting enzyme (ICE). The two proteins share 28% identity at the amino acid level, and ced-3 is identical to ICE protein in a five amino acid stretch thought to be the active site responsible for ICE's protease activity.

Apoptosis may be induced by a variety of different extracellular and intracellular stimuli, some of which are still unknown, which can differ depending on cell type. However, the numerous receptors and associated signal transduction pathways that respond to each different induction stimulus may converge on one or more of a limited number of pathways that actually “trigger” the program for apoptotic cell death. Among the stimuli which can induce apoptosis, there is extracellular ATP, actinomycin and oxygen radicals. Actinomycin inhibits the synthesis of RNA and induces apoptosis in some mammalian cell types, including human primary uterine epithelial cells and HL60 leukemic cells (Gerschenson, L. E. and Rotello, R. J., Cold Spring Harbor, Laboratory Press, Cold Spring Harbor, N.Y., page 175, (1991) and Martin, S. J., et al., Immunol., 145:1859 (1990)).

The baculovirus, Cydia pomonella granulosis virus (CpGV), was able to inhibit apoptosis in SF-21 cells (derived from the fall army worm Spodoptera frugiperda) from a mutated baculovirus AcMNPV which causes apoptosis in SF-21 cells in the absence of CpGV. The CpGV gene was sequenced (Crook, N. E. et al., J. Virol., 67:2168 (1993)) and found to have a characteristic zinc finger-like motif. The gene was named iap for inhibitor of apoptosis, and the CpGV gene was called the Cp-IAP.

The zinc finger-like motif found in the Cp-IAP polypeptide belongs to a specific class of zinc finger-like motifs (Freemont, P. S. et al., Cell, 64:483 (1991)) usually found at the amino terminus of polypeptides, but it can occur elsewhere, as in the case of the IAP polypeptide, where it is found at the carboxyl terminus. The IAP motif also contains an additional CX C repeat in the amino-terminal portion of the motif, as well as an extra amino acid residue in the central region (CXHX C instead of CXHX C). There are approximately 27 known proteins containing this type of zinc finger-like motif, four are found in baculoviruses. The zinc finger-like motif in also present in several proteins encoded by vertebrate viruses. The presence of the motif in several regulatory proteins supports the hypothesis that many of the proteins containing this motif may be transcriptional regulatory factors, although DNA binding has been demonstrated thus far for only one particular member of this group (Tagawa, et al., J. Biol. Chem., 265:20021 (1990)).

The zinc finger-like motif found in Cp-IAP is also present in a number of cellular polypeptides that may have a role in regulating apoptosis. Several of these are encoded by human proto-oncogenes such as PML, bmi-1, c-cbl, rfp and mel-18.

These proteins are involved in either positive or negative apoptotic control. Thus, Cp-IAP may belong to a class of cellular proteins which control apoptosis and contain this distinctive zinc finger-like motif. The polypeptide of the present invention contains the conserved zinc finger-like motifs and has been putatively identified as a member of this class of proteins.

Apoptosis has been shown to play a significant role in cell development, antiviral responses, tissue differentiation, development, tissue homeostasis, Alzheimer's disease, rheumatoid arthritis, septic shock, Parkinson's disease and may be a mechanism by which cells die during strokes, trauma and degenerative diseases. Improper apoptosis, i.e., when cells fail to die when they should, may result in tumors, oncogenesis and viral infection.

In accordance with one aspect of the present invention, there is provided a novel mature polypeptide as well as biologically active and diagnostically or therapeutically useful fragments, analogs and derivatives thereof. The polypeptide of the present invention is of human origin.

In accordance with another aspect of the present invention, there are provided isolated nucleic acid molecules encoding a polypeptide of the present invention including mRNAs, DNAs, cDNAs, genomic DNAs as well as analogs and biologically active and diagnostically or therapeutically useful fragments and derivatives thereof.

In accordance with yet a further aspect of the present invention, there is provided a process for producing such polypeptide by recombinant techniques comprising culturing recombinant prokaryotic and/or eukaryotic host cells, containing a nucleic acid sequence, under conditions promoting expression of said protein and subsequent recovery of said protein.

In accordance with yet a further aspect of the present invention, there is provided a process for utilizing such polypeptide, or polynucleotide encoding such polypeptide for therapeutic purposes, for example, for preventing oncogenesis, to treat Alzheimer's disease, Parkinson's disease, rheumatoid arthritis, septic shock and to prevent the death of cells during strokes, trauma and degenerative diseases.

In accordance with yet a further aspect of the present invention, there is also provided nucleic acid probes comprising nucleic acid molecules of sufficient length to specifically hybridize to nucleic acid sequences of the present invention.

In accordance with yet a further aspect of the present invention, there are provided antibodies against such polypeptides.

In accordance with yet another aspect of the present invention, there are provided compounds which bind to and inhibit such polypeptides, which may be used, for example, as an anti-viral defense mechanism, to allow cell development, tissue differentiation, tissue homeostasis and normal development.

In accordance with still another aspect of the present invention, there are provided processes for employing the disclosed polynucleotides and polypeptides for research purposes.

These and other aspects of the present invention should be apparent to those skilled in the art from the teachings herein.

### BRIEF DESCRIPTION OF THE DRAWINGS

The following drawings are illustrative of embodiments of the invention and are not meant to limit the scope of the invention as encompassed by the claims.

FIG. 1 illustrates the cDNA sequence (SEQ ID NO:1) and corresponding deduced amino acid sequence (SEQ ID NO:2) of the polypeptide of the present invention. The standard abbreviations for nucleotides and amino acids are used. Sequencing was performed using a 373 automated DNA sequencer (Applied Biosystems, Inc.).

FIGS. 2A-2B is an amino acid sequence alignment of hIAP-1 with Cp-IAP (Cydia pomonella granulosis virus inhibitor of apoptosis, SEQ ID NO:9) and Op-IAP (Orgyia pseudotsugata nuclear polyhedrosis virus inhibitor of apoptosis, SEQ ID NO:10).

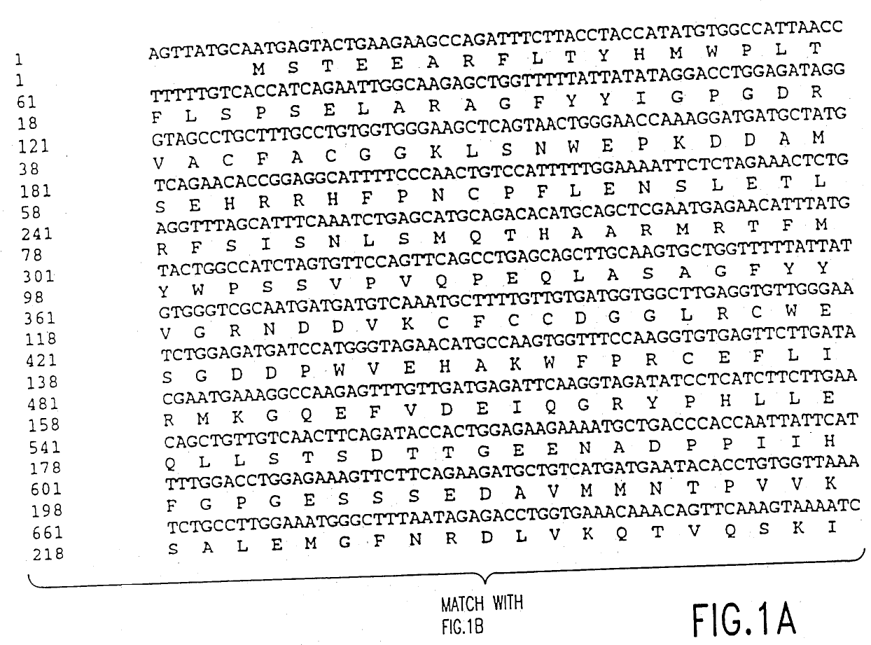

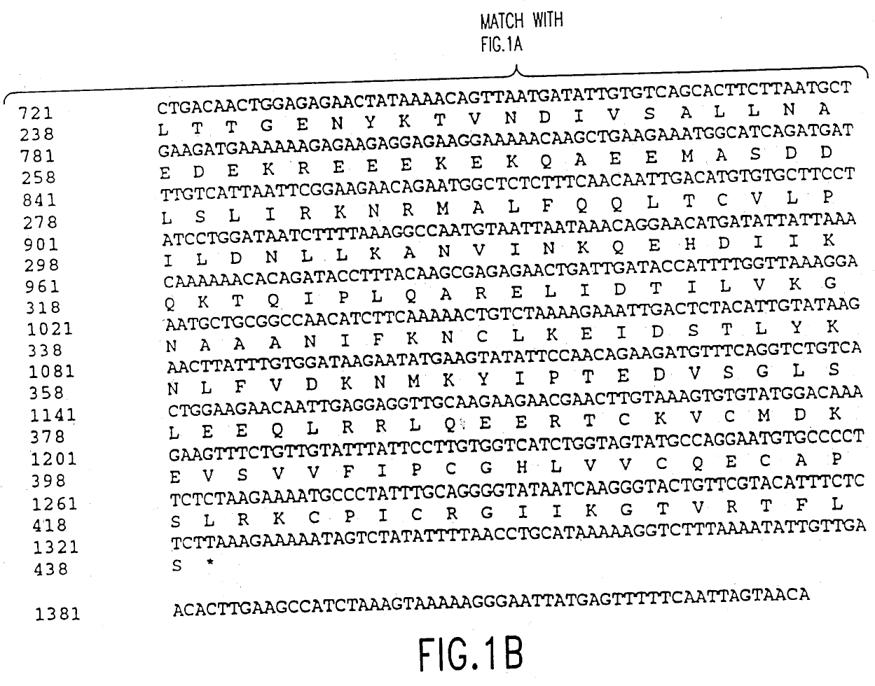

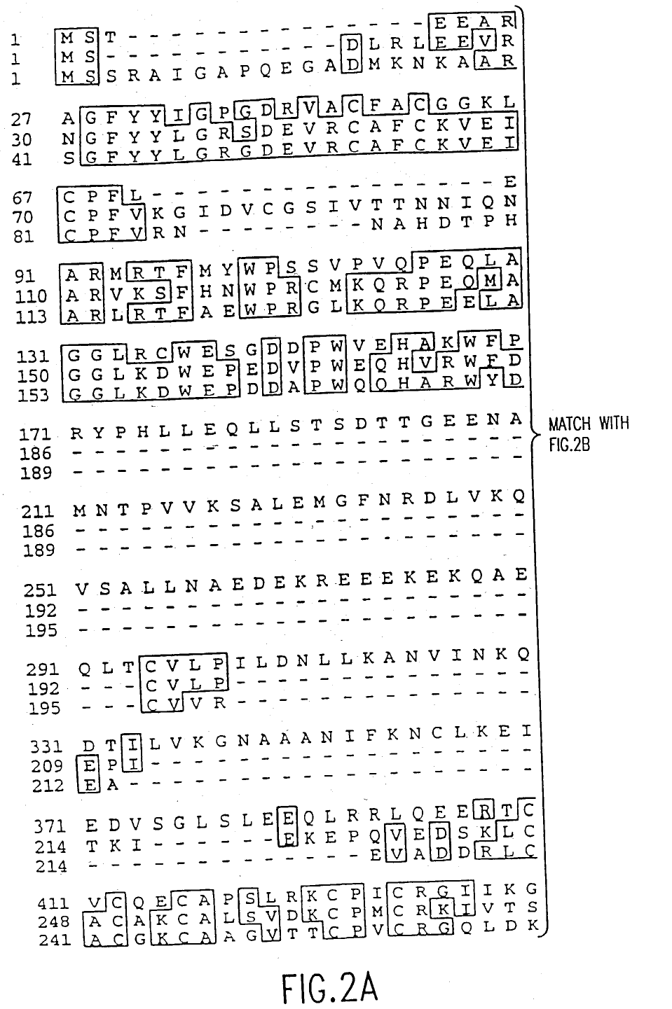

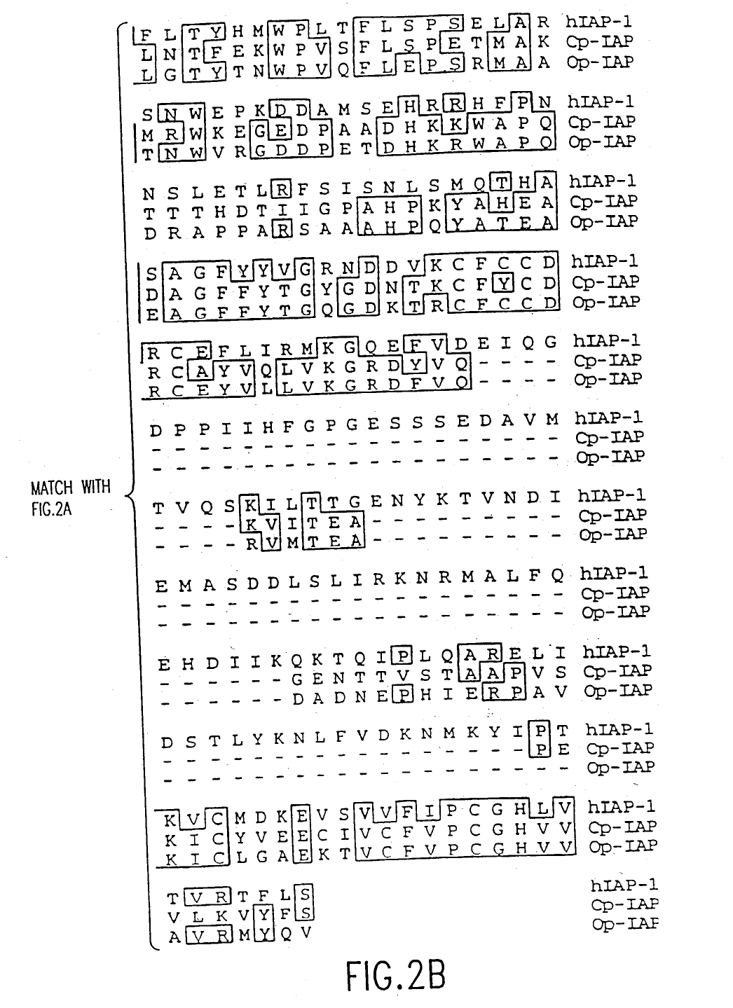

In accordance with an aspect of the present invention, there is provided an isolated nucleic acid (polynucleotide) which encodes for the mature polypeptide having the deduced amino acid sequence of FIGS. 1A-1B (SEQ ID NO:2) or for the mature polypeptide encoded by the cDNA of the clone deposited as ATCC Deposit No. 97141 on May 11, 1995.

The ATCC deposit referred to above was deposited with the ATCC, 10801 University Blvd., Manssas Va. 20110-2209, USA. Since the strain referred to is being maintained under the terms of the Budapest Treaty, it will be made available to a patent office signatory to the Budapest Treaty.

A polynucleotide encoding a polypeptide of the present invention may be obtained from human jurket cell lines and human osteoclastoma stromal cells. It is structurally related to the inhibitor of apoptosis gene family. It contains an open reading frame encoding a protein of 438 amino acid residues. The protein exhibits the highest degree of homology to Op-IAP with 44% identity and 64% similarity over the entire amino acid stretch. As stated previously, the conserved motifs found in genes of this type are preserved in the gene of the present invention.

The polynucleotide of the present invention may be in the form of RNA or in the form of DNA, which DNA includes cDNA, genomic DNA, and synthetic DNA. The DNA may be double-stranded or single-stranded, and if single stranded may be the coding strand or non-coding (anti-sense) strand. The coding sequence which encodes the mature polypeptide may be identical to the coding sequence shown in FIGS. 1A-1B (SEQ ID NO:1) or that of the deposited clone or may be a different coding sequence which coding sequence, as a result of the redundancy or degeneracy of the genetic code, encodes the same mature polypeptide as the DNA of FIGS. 1A-1B (SEQ ID NO:1) or the deposited cDNA.

The polynucleotide which encodes for the mature polypeptide of FIGS. 1A-1B (SEQ ID NO:2) or for the mature polypeptide encoded by the deposited cDNA may include, but is not limited to: only the coding sequence for the mature polypeptide; the coding sequence for the mature polypeptide and additional coding sequence; the coding sequence for the mature polypeptide (and optionally additional coding sequence) and non-coding sequence, such as introns or non-coding sequence 5′ and/or 3′ of the coding sequence for the mature polypeptide.

Thus, the term “polynucleotide encoding a polypeptide” encompasses a polynucleotide which includes only coding sequence for the polypeptide as well as a polynucleotide which includes additional coding and/or non-coding sequence.

The present invention further relates to variants of the hereinabove described polynucleotides which encode for fragments, analogs and derivatives of the polypeptide having the deduced amino acid sequence of FIGS. 1A-1B (SEQ ID NO:2) or the polypeptide encoded by the cDNA of the deposited clone. The variant of the polynucleotide may be a naturally occurring allelic variant of the polynucleotide or a non-naturally occurring variant of the polynucleotide.

Thus, the present invention includes polynucleotides encoding the same mature polypeptide as shown in FIGS. 1A-1B (SEQ ID NO:2) or the same mature polypeptide encoded by the cDNA of the deposited clone as well as variants of such polynucleotides which variants encode for a fragment, derivative or analog of the polypeptide of FIGS. 1A-1B (SEQ ID NO:2) or the polypeptide encoded by the cDNA of the deposited clone. Such nucleotide variants include deletion variants, substitution variants and addition or insertion variants.

As hereinabove indicated, the polynucleotide may have a coding sequence which is a naturally occurring allelic variant of the coding sequence shown in FIG. 1 (SEQ ID NO:1) or of the coding sequence of the deposited clone. As known in the art, an allelic variant is an alternate form of a polynucleotide sequence which may have a substitution, deletion or addition of one or more nucleotides, which does not substantially alter the function of the encoded polypeptide.

The polynucleotides of the present invention may also have the coding sequence fused in frame to a marker sequence which allows for purification of the polypeptide of the present invention. The marker sequence may be a hexa-histidine tag supplied by a pQE-9 vector to provide for purification of the mature polypeptide fused to the marker in the case of a bacterial host, or, for example, the marker sequence may be a hemagglutinin (HA) tag when a mammalian host, e.g. COS-7 cells, is used. The HA tag corresponds to an epitope derived from the influenza hemagglutinin protein (Wilson, I., et al., Cell, 37:767 (1984)).

The term “gene” means the segment of DNA involved in producing a polypeptide chain; it includes regions preceding and following the coding region (leader and trailer) as well as intervening sequences (introns) between individual coding segments (exons).

Fragments of the full length gene of the present invention may be used as a hybridization probe for a cDNA library to isolate the full length cDNA and to isolate other cDNAs which have a high sequence similarity to the gene or similar biological activity. Probes of this type preferably have at least 30 bases and may contain, for example, 50 or more bases. The probe may also be used to identify a cDNA clone corresponding to a full length transcript and a genomic clone or clones that contain the complete gene including regulatory and promotor regions, exons, and introns. An example of a screen comprises isolating the coding region of the gene by using the known DNA sequence to synthesize an oligonucleotide probe. Labeled oligonucleotides having a sequence complementary to that of the gene of the present invention are used to screen a library of human cDNA, genomic DNA or mRNA to determine which members of the library the probe hybridizes to.

The present invention further relates to polynucleotides which hybridize to the hereinabove-described sequences if there is at least 70%, preferably at least 90%, and more preferably at least 95% identity between the sequences. The present invention particularly relates to polynucleotides which hybridize under stringent conditions to the hereinabove-described polynucleotides. As herein used, the term “stringent conditions” means hybridization will occur only if there is at least 95% and preferably at least 97% identity between the sequences. The polynucleotides which hybridize to the hereinabove described polynucleotides in a preferred embodiment encode polypeptides which either retain substantially the same biological function or activity as the mature polypeptide encoded by the cDNAs of FIGS. 1A-1B (SEQ ID NO:1) or the deposited cDNA(s).

Alternatively, the polynucleotides may have at least 20 bases, preferably 30 bases and more preferably at least 50 bases which hybridize to a polynucleotide of the present invention and which have an identity thereto, as hereinabove described, which may or may not retain activity. Such polynucleotides may be employed as probes for the polynucleotide of SEQ ID NO: 1, or for variants thereof, for example, for recovery of the polynucleotide or as a diagnostic probe or as a PCR primer.

Thus, the present invention is directed to polynucleotides having at least a 70% identity, preferably at least 90% and more preferably at least a 95% identity to a polynucleotide which encodes the polypeptide of SEQ ID NO:2 as well as fragments thereof, which fragments have at least 30 bases and preferably at least 50 bases and to polypeptides encoded by such polynucleotides.

The deposit(s) referred to herein will be maintained under the terms of the Budapest Treaty on the International Recognition of the Deposit of Micro-organisms for purposes of Patent Procedure. These deposits are provided merely as convenience to those of skill in the art and are not an admission that a deposit is required under 35 U.S.C. §112. The sequence of the polynucleotides contained in the deposited materials, as well as the amino acid sequence of the polypeptides encoded thereby, are incorporated herein by reference and are controlling in the event of any conflict with any description of sequences herein. A license may be required to make, use or sell the deposited materials, and no such license is hereby granted.

The present invention further relates to a polypeptide which has the deduced amino acid sequence of FIGS. 1A-1B (SEQ ID NO:2) or which has the amino acid sequence encoded by the deposited cDNA, as well as fragments, analogs and derivatives of such polypeptide.

The terms “fragment,” “derivative” and “analog” when referring to the polypeptide of FIGS. 1A-1B (SEQ ID NO:2) or that encoded by the deposited cDNA, means a polypeptide which retains essentially the same biological function or activity as such polypeptide. Thus, an analog includes a proprotein which can be activated by cleavage of the proprotein portion to produce an active mature polypeptide.

The polypeptide of the present invention may be a recombinant polypeptide, a natural polypeptide or a synthetic polypeptide, preferably a recombinant polypeptide.

The fragment, derivative or analog of the polypeptide of FIGS. 1A-1B (SEQ ID NO:2) or that encoded by the deposited cDNA may be (i) one in which one or more of the amino acid residues are substituted with a conserved or non-conserved amino acid residue (preferably a conserved amino acid residue) and such substituted amino acid residue may or may not be one encoded by the genetic code, or (ii) one in which one or more of the amino acid residues includes a substituent group, or (iii) one in which the mature polypeptide is fused with another compound, such as a compound to increase the half-life of the polypeptide (for example, polyethylene glycol), or (iv) one in which the additional amino acids are fused to the mature polypeptide which are employed for purification of the mature polypeptide or a proprotein sequence. Such fragments, derivatives and analogs are deemed to be within the scope of those skilled in the art from the teachings herein.

The polypeptides and polynucleotides of the present invention are preferably provided in an isolated form, and preferably are purified to homogeneity.

The term “isolated” means that the material is removed from its original environment (e.g., the natural environment if it is naturally occurring). For example, a naturally-occurring polynucleotide or polypeptide present in a living animal is not isolated, but the same polynucleotide or polypeptide, separated from some or all of the coexisting materials in the natural system, is isolated. Such polynucleotides could be part of a vector and/or such polynucleotides or polypeptides could be part of a composition, and still be isolated in that such vector or composition is not part of its natural environment.

The polypeptides of the present invention include the polypeptide of SEQ ID NO:2 (in particular the mature polypeptide) as well as polypeptides which have at least 70% similarity (preferably at least 70% identity) to the polypeptide of SEQ ID NO:2 and more preferably at least 90% similarity (more preferably at least 90% identity) to the polypeptide of SEQ ID NO:2 and still more preferably at least 95% similarity (still more preferably at least 90% identity) to the polypeptide of SEQ ID NO:2 and also include portions of such polypeptides with such portion of the polypeptide generally containing at least 30 amino acids and more preferably at least 50 amino acids.

As known in the art “similarity” between two polypeptides is determined by comparing the amino acid sequence and its conserved amino acid substitutes of one polypeptide to the sequence of a second polypeptide.

Fragments or portions of the polypeptides of the present invention may be employed for producing the corresponding full-length polypeptide by peptide synthesis; therefore, the fragments may be employed as intermediates for producing the full-length polypeptides. Fragments or portions of the polynucleotides of the present invention may be used to synthesize full-length polynucleotides of the present invention.

The present invention also relates to vectors which include polynucleotides of the present invention, host cells which are genetically engineered with vectors of the invention and the production of polypeptides of the invention by recombinant techniques.

Host cells are genetically engineered (transduced or transformed or transfected) with the vectors of this invention which may be, for example, a cloning vector or an expression vector. The vector may be, for example, in the form of a plasmid, a viral particle, a phage, etc. The engineered host cells can be cultured in conventional nutrient media modified as appropriate for activating promoters, selecting transformants or amplifying the hIAP-1 genes. The culture conditions, such as temperature, pH and the like, are those previously used with the host cell selected for expression, and will be apparent to the ordinarily skilled artisan.

The polynucleotides of the present invention may be employed for producing polypeptides by recombinant techniques. Thus, for example, the polynucleotide may be included in any one of a variety of expression vectors for expressing a polypeptide. Such vectors include chromosomal, nonchromosomal and synthetic DNA sequences, e.g., derivatives of SV40; bacterial plasmids; phage DNA; baculovirus; yeast plasmids; vectors derived from combinations of plasmids and phage DNA, viral DNA such as vaccinia, adenovirus, fowl pox virus, and pseudorabies. However, any other vector may be used as long as it is replicable and viable in the host.

The appropriate DNA sequence may be inserted into the vector by a variety of procedures. In general, the DNA sequence is inserted into an appropriate restriction endonuclease site(s) by procedures known in the art. Such procedures and others are deemed to be within the scope of those skilled in the art.

The DNA sequence in the expression vector is operatively linked to an appropriate expression control sequence(s) (promoter) to direct mRNA synthesis. As representative examples of such promoters, there may be mentioned: LTR or SV40 promoter, the E. coli. lac or trp, the phage lambda P promoter and other promoters known to control expression of genes in prokaryotic or eukaryotic cells or their viruses. The expression vector also contains a ribosome binding site for translation initiation and a transcription terminator. The vector may also include appropriate sequences for amplifying expression.

In addition, the expression vectors preferably contain one or more selectable marker genes to provide a phenotypic trait for selection of transformed host cells such as dihydrofolate reductase or neomycin resistance for eukaryotic cell culture, or such as tetracycline or ampicillin resistance in E. coli.

The vector containing the appropriate DNA sequence as hereinabove described, as well as an appropriate promoter or control sequence, may be employed to transform an appropriate host to permit the host to express the protein.

As representative examples of appropriate hosts, there may be mentioned: bacterial cells, such as E. coli, Streptomyces, Salmonella typhimurium; fungal cells, such as yeast; insect cells such as Drosophila S2 and Spodoptera Sf9; animal cells such as CHO, COS or Bowes melanoma; adenoviruses; plant cells, etc. The selection of an appropriate host is deemed to be within the scope of those skilled in the art from the teachings herein.

More particularly, the present invention also includes recombinant constructs comprising one or more of the sequences as broadly described above. The constructs comprise a vector, such as a plasmid or viral vector, into which a sequence of the invention has been inserted, in a forward or reverse orientation. In a preferred aspect of this embodiment, the construct further comprises regulatory sequences, including, for example, a promoter, operably linked to the sequence. Large numbers of suitable vectors and promoters are known to those of skill in the art, and are commercially available. The following vectors are provided by way of example; Bacterial: pQE70, pQE60, pQE-9 (Qiagen), pBS, pD10, phagescript, psiX174, pbluescript SK, pbsks, pNH8A, pNH16a, pNH18A, pNH46A (Stratagene); ptrc99a, pKK223-3, pKK233-3, pDR540, pRIT5 (Pharmacia); Eukaryotic: pWLNEO, pSV2CAT, pOG44, pXT1, pSG (Stratagene) pSVK3, pBPV, pMSG, pSVL (Pharmacia). However, any other plasmid or vector may be used as long as they are replicable and viable in the host.

Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P, P and trp. Eukaryotic promoters include CMV immediate early, HSV thymidine kinase, early and late SV40, LTRs from retrovirus, and mouse metallothionein-I. Selection of the appropriate vector and promoter is well within the level of ordinary skill in the art.

In a further embodiment, the present invention relates to host cells containing the above-described constructs. The host cell can be a higher eukaryotic cell, such as a mammalian cell, or a lower eukaryotic cell, such as a yeast cell, or the host cell can be a prokaryotic cell, such as a bacterial cell. Introduction of the construct into the host cell can be effected by calcium phosphate transfection, DEAE-Dextran mediated transfection, or electroporation (Davis, L., Dibner, M., Battey, I., Basic Methods in Molecular Biology, (1986)).

The constructs in host cells can be used in a conventional manner to produce the gene product encoded by the recombinant sequence. Alternatively, the polypeptides of the invention can be synthetically produced by conventional peptide synthesizers.

Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., Molecular Cloning: A Laboratory Manual, Second Edition, Cold Spring Harbor, N.Y., (1989), the disclosure of which is hereby incorporated by reference.

Transcription of the DNA encoding the polypeptides of the present invention by higher eukaryotes is increased by inserting an enhancer sequence into the vector. Enhancers are cis-acting elements of DNA, usually about from 10 to 300 bp that act on a promoter to increase its transcription. Examples including the SV40 enhancer on the late side of the replication origin bp 100 to 270, a cytomegalovirus early promoter enhancer, the polyoma enhancer on the late side of the replication origin, and adenovirus enhancers.

Generally, recombinant expression vectors will include origins of replication and selectable markers permitting transformation of the host cell, e.g., the ampicillin resistance gene of E. coli and S. cerevisiae TRP1 gene, and a promoter derived from a highly-expressed gene to direct transcription of a downstream structural sequence. Such promoters can be derived from operons encoding glycolytic enzymes such as 3-phosphoglycerate kinase (PGK), Â-factor, acid phosphatase, or heat shock proteins, among others. The heterologous structural sequence is assembled in appropriate phase with translation initiation and termination sequences. Optionally, the heterologous sequence can encode a fusion protein including an N-terminal identification peptide imparting desired characteristics, e.g., stabilization or simplified purification of expressed recombinant product.

Useful expression vectors for bacterial use are constructed by inserting a structural DNA sequence encoding a desired protein together with suitable translation initiation and termination signals in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but nonlimiting example, useful expression vectors for bacterial use can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and GEMI (Promega Biotec, Madison, Wis., USA). These pBR322 “backbone” sections are combined with an appropriate promoter and the structural sequence to be expressed.

Following transformation of a suitable host strain and growth of the host strain to an appropriate cell density, the selected promoter is induced by appropriate means (e.g., temperature shift or chemical induction) and cells are cultured for an additional period.

Cells are typically harvested by centrifugation, disrupted by physical or chemical means, and the resulting crude extract retained for further purification.

Microbial cells employed in expression of proteins can be disrupted by any convenient method, including freeze-thaw cycling, sonication, mechanical disruption, or use of cell lysing agents, such methods are well know to those skilled in the art.

Various mammalian cell culture systems can also be employed to express recombinant protein. Examples of mammalian expression systems include the COS-7 lines of monkey kidney fibroblasts, described by Gluzman, Cell, 23:175 (1981), and other cell lines capable of expressing a compatible vector, for example, the C127, 3T3, CHO, HeLa and BHK cell lines. Mammalian expression vectors will comprise an origin of replication, a suitable promoter and enhancer, and also any necessary ribosome binding sites, polyadenylation site, splice donor and acceptor sites, transcriptional termination sequences, and 5′ flanking nontranscribed sequences. DNA sequences derived from the SV40 splice, and polyadenylation sites may be used to provide the required nontranscribed genetic elements.

The polypeptide can be recovered and purified from recombinant cell cultures by methods including ammonium sulfate or ethanol precipitation, acid extraction, anion or cation exchange chromatography, phosphocellulose chromatography, hydrophobic interaction chromatography, affinity chromatography, hydroxylapatite chromatography and lectin chromatography. Protein refolding steps can be used, as necessary, in completing configuration of the mature protein. Finally, high performance liquid chromatography (HPLC) can be employed for final purification steps.

The polypeptides of the present invention may be a naturally purified product, or a product of chemical synthetic procedures, or produced by recombinant techniques from a prokaryotic or eukaryotic host (for example, by bacterial, yeast, higher plant, insect and mammalian cells in culture). Depending upon the host employed in a recombinant production procedure, the polypeptides of the present invention may be glycosylated or may be non-glycosylated. Polypeptides of the invention may also include an initial methionine amino acid residue.

The hIAP-1 polypeptides and inhibitory compounds, described below, which are polypeptides may also be employed in accordance with the present invention by expression of such polypeptides in vivo, which is referred to as “gene therapy.”

Thus, for example, cells from a patient may be engineered with a polynucleotide (DNA or RNA) encoding a polypeptide ex vivo, with the engineered cells then being provided to a patient to be treated with the polypeptide. Such methods are well-known in the art and are apparent from the teachings herein. For example, cells may be engineered by the use of a retroviral plasmid vector containing RNA encoding a polypeptide of the present invention.

Similarly, cells may be engineered in vivo for expression of a polypeptide in vivo by, for example, procedures known in the art. For example, a packaging cell is transduced with a retroviral plasmid vector containing RNA encoding a polypeptide of the present invention such that the packaging cell now produces infectious viral particles containing the gene of interest. These producer cells may be administered to a patient for engineering cells in vivo and expression of the polypeptide in vivo. These and other methods for administering a polypeptide of the present invention by such method should be apparent to those skilled in the art from the teachings of the present invention.

Retroviruses from which the retroviral plasmid vectors hereinabove mentioned may be derived include, but are not limited to, Moloney Murine Leukemia Virus, spleen necrosis virus, retroviruses such as Rous Sarcoma Virus, Harvey Sarcoma Virus, avian leukosis virus, gibbon ape leukemia virus, human immunodeficiency virus, adenovirus, Myeloproliferative Sarcoma Virus, and mammary tumor virus. In one embodiment, the retroviral plasmid vector is derived from Moloney Murine Leukemia Virus.

The vector includes one or more promoters. Suitable promoters which may be employed include, but are not limited to, the retroviral LTR; the SV40 promoter; and the human cytomegalovirus (CMV) promoter described in Miller, et al., Biotechniques, Vol. 7, NO:9, 980-990 (1989), or any other promoter (e.g., cellular promoters such as eukaryotic cellular promoters including, but not limited to, the histone, pol III, and -actin promoters). Other viral promoters which may be employed include, but are not limited to, adenovirus promoters, thymidine kinase (TK) promoters, and B19 parvovirus promoters. The selection of a suitable promoter will be apparent to those skilled in the art from the teachings contained herein.

The nucleic acid sequence encoding the polypeptide of the present invention is under the control of a suitable promoter. Suitable promoters which may be employed include, but are not limited to, adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs (including the modified retroviral LTRs hereinabove described); the -actin promoter; and human growth hormone promoters. The promoter also may be the native promoter which controls the gene encoding the polypeptide.

The retroviral plasmid vector is employed to transduce packaging cell lines to form producer cell lines. Examples of packaging cells which may be transfected include, but are not limited to, the PE501, PA317, -2, -AM, PA12, T19-14X, VT-19-17-H2, CRE, CRIP, GP+E-86, GP+envAm12, and DAN cell lines as described in Miller, Human Gene Therapy, Vol. 1, pgs. 5-14 (1990), which is incorporated herein by reference in its entirety. The vector may transduce the packaging cells through any means known in the art. Such means include, but are not limited to, electroporation, the use of liposomes, and CaPO precipitation. In one alternative, the retroviral plasmid vector may be encapsulated into a liposome, or coupled to a lipid, and then administered to a host.

The producer cell line generates infectious retroviral vector particles which include the nucleic acid sequence(s) encoding the polypeptides. Such retroviral vector particles then may be employed, to transduce eukaryotic cells, either in vitro or in vivo. The transduced eukaryotic cells will express the nucleic acid sequence(s) encoding the polypeptide. Eukaryotic cells which may be transduced include, but are not limited to, embryonic stem cells, embryonic carcinoma cells, as well as hematopoietic stem cells, hepatocytes, fibroblasts, myoblasts, keratinocytes, endothelial cells, and bronchial epithelial cells.

Once the hIAP-1 polypeptide is being expressed intra-cellularly via gene therapy, it may be employed to treat neurodegenerative diseases caused by abnormal apoptosis of neurons, for example, Alzheimer's disease and Parkinson's disease.

hIAP-1 may also be employed to prevent cells from dying during trauma such as head injury and strokes.

The hIAP-1 protein of the present invention may also be employed to prevent abnormal apoptosis which leads to rheumatoid arthritis.

hIAP-1 polypeptide may also be employed to prevent oncogenesis which results from abnormal apoptosis.

In accordance with yet a further aspect of the present invention, there is provided a process for utilizing such polypeptides, or polynucleotides encoding such polypeptides, for in vitro purposes related to scientific research, synthesis of DNA and manufacture of DNA vectors. For example, the gene and gene product may be employed as a research tool for discovering diagnostic and therapeutic treatments for human disease and to shed light on the process of apoptosis in humans.

This invention is also related to the use of the hIAP-1 gene as a diagnostic. Detection of a mutated form of hIAP-1 will allow a diagnosis of a disease or a susceptibility to a disease which results from underexpression of hIAP-1.

Individuals carrying mutations in the human hIAP-1 gene may be detected at the DNA level by a variety of techniques. Nucleic acids for diagnosis may be obtained from a patient's cells, such as from blood, urine, saliva, tissue biopsy and autopsy material. The genomic DNA may be used directly for detection or may be amplified enzymatically by using PCR (Saiki et al., Nature, 324:163-166 (1986)) prior to analysis. RNA or cDNA may also be used for the same purpose. As an example, PCR primers complementary to the nucleic acid encoding hIAP-1 can be used to identify and analyze hIAP-1 mutations. For example, deletions and insertions can be detected by a change in size of the amplified product in comparison to the normal genotype. Point mutations can be identified by hybridizing amplified DNA to radiolabeled hIAP-1 RNA or alternatively, radiolabeled hIAP-1 antisense DNA sequences. Perfectly matched sequences can be distinguished from mismatched duplexes by RNase A digestion or by differences in melting temperatures.

Sequence differences between the reference gene and genes having mutations may be revealed by the direct DNA sequencing method. In addition, cloned DNA segments may be employed as probes to detect specific DNA segments. The sensitivity of this method is greatly enhanced when combined with PCR. For example, a sequencing primer is used with double-stranded PCR product or a single-stranded template molecule generated by a modified PCR. The sequence determination is performed by conventional procedures with radiolabeled nucleotide or by automatic sequencing procedures with fluorescent-tags.

Genetic testing based on DNA sequence differences may be achieved by detection of alteration in electrophoretic mobility of DNA fragments in gels with or without denaturing agents. Small sequence deletions and insertions can be visualized by high resolution gel electrophoresis. DNA fragments of different sequences may be distinguished on denaturing formamide gradient gels in which the mobilities of different DNA fragments are retarded in the gel at different positions according to their specific melting or partial melting temperatures (see, e.g., Myers et al., Science, 230:1242 (1985)).

Sequence changes at specific locations may also be revealed by nuclease protection assays, such as RNase and S1 protection or the chemical cleavage method (e.g., Cotton et al., PNAS, USA, 85:4397-4401 (1985)).

Thus, the detection of a specific DNA sequence may be achieved by methods such as hybridization, RNase protection, chemical cleavage, direct DNA sequencing or the use of restriction enzymes, (e.g., Restriction Fragment Length Polymorphisms (RFLP)) and Southern blotting of genomic DNA.

In addition to more conventional gel-electrophoresis and DNA sequencing, mutations can also be detected by in situ analysis.

The present invention also relates to a diagnostic assay for detecting altered levels of hIAP-1 protein in various tissues since an over-expression of the proteins compared to normal control tissue samples can detect the presence of conditions related to abnormally high apoptosis. Assays used to detect levels of hIAP-1 protein in a sample derived from a host are well-known to those of skill in the art and include radioimmunoassays, competitive-binding assays, Western Blot analysis and preferably an ELISA assay. An ELISA assay initially comprises preparing an antibody specific to the hIAP-1 antigen, preferably a monoclonal antibody. In addition a reporter antibody is prepared against the monoclonal antibody. To the reporter antibody is attached a detectable reagent such as radioactivity, fluorescence or in this example a horseradish peroxidase enzyme. A sample is now removed from a host and incubated on a solid support, e.g. a polystyrene dish, that binds the proteins in the sample. Any free protein binding sites on the dish are then covered by incubating with a non-specific protein like BSA. Next, the monoclonal antibody is incubated in the dish during which time the monoclonal antibodies attach to any hIAP-1 proteins attached to the polystyrene dish. All unbound monoclonal antibody is washed out with buffer. The reporter antibody linked to horseradish peroxidase is now placed in the dish resulting in binding of the reporter antibody to any monoclonal antibody bound to hIAP-1. Unattached reporter antibody is then washed out. Peroxidase substrates are then added to the dish and the amount of color developed in a given time period is a measurement of the amount of hIAP-1 protein present in a given volume of patient sample when compared against a standard curve.

A competition assay may be employed wherein antibodies specific to hIAP-1 is attached to a solid support and labeled hIAP-1 and a sample derived from the host are passed over the solid support and the amount of label detected attached to the solid support can be correlated to a quantity of hIAP-1 in the sample.

This invention provides a method of screening compounds to identify those which block the inhibition of apoptosis by hIAP-1. For example, SF-21 cells are transfected with a known gene which causes apoptosis, for example, annihilator mutant DNA from vAcAnh or vP35Z. The compound to be tested and hIAP are then contacted with the cell, either intracellularly or extracellularly. A survey is then done of the cells under a light microscope three to four days after co-transfection, and the usual characteristics of an apoptotic cell is checked and the ability of the compound to prevent the action of hIAP is analyzed.

Human IAP-1 is produced and functions intra-cellularly, therefore, any inhibitory compounds must function intra-cellularly. These compounds include antibodies which are produced intracellularly. For example, an antibody identified as inhibiting hIAP-1 may be produced intracellularly as a single chain antibody by procedures known in the art, such as transforming the appropriate cells with DNA encoding the single chain antibody to prevent the function of hIAP-1.

Another example is an antisense construct prepared using antisense technology used to control gene expression through triple-helix formation or antisense DNA or RNA, both of which methods are based on binding of a polynucleotide to DNA or RNA. For example, the 5′ coding portion of the polynucleotide sequence, which encodes for the mature polypeptides of the present invention, is used to design an antisense RNA oligonucleotide of from about 10 to 40 base pairs in length. A DNA oligonucleotide is designed to be complementary to a region of the gene involved in transcription (triple helix—see Lee et al., Nucl. Acids Res., 6:3073 (1979); Cooney et al, Science, 241:456 (1988); and Dervan et al., Science, 251: 1360 (1991)), thereby preventing transcription and the production of hIAP-1. The antisense RNA oligonucleotide hybridizes to the mRNA in vivo and blocks translation of the MRNA molecule into hIAP-1 polypeptide (Antisense—Okano, J. Neurochem., 56:560 (1991); Oligodeoxynucleotides as Antisense Inhibitors of Gene Expression, CRC Press, Boca Raton, Fla. (1988)). The oligonucleotides described above can also be delivered to cells such that the antisense RNA or DNA may be expressed in vivo to inhibit production of hIAP-1.

Another example includes a mutated form, or mutein, of hIAP-1 which recognizes hIAP-1 substrates but has impaired function so as not to prevent apoptosis.

Another example is a small molecule which is able to pass through the cell membrane and bind to hIAP-1 and prevent its biological activity. Examples of small molecules include but are not limited to small peptides or peptide-like molecules.

These compounds may be employed to inhibit the action of hIAP-1 and prevent tumors since oncogenesis results when cells fail to undergo apoptosis at the appropriate time.

The action of hIAP-1 may also be inhibited by these compounds for the promotion of cell development to allow apoptosis to kill viral infected cells, and to stimulate tissue differentiation and development. The compounds may also be employed to maintain tissue homeostasis.

The compounds may be employed in combination with a suitable pharmaceutical carrier. Such compositions comprise a therapeutically effective amount of the compound, and a pharmaceutically acceptable carrier or excipient. Such a carrier includes but is not limited to saline, buffered saline, dextrose, water, glycerol, ethanol, and combinations thereof. The formulation should suit the mode of administration.

The invention also provides a pharmaceutical pack or kit comprising one or more containers filled with one or more of the ingredients of the pharmaceutical compositions of the invention. Associated with such container(s) can be a notice in the form prescribed by a governmental agency regulating the manufacture, use or sale of pharmaceuticals or biological products, which notice reflects approval by the agency of manufacture, use or sale for human administration. In addition, the compounds may be employed in conjunction with other therapeutic compounds.

The pharmaceutical compositions may be administered in a convenient manner such as by the oral, topical, intravenous, intraperitoneal, intramuscular, subcutaneous, intranasal or intradermal routes. The pharmaceutical compositions are administered in an amount which is effective for treating and/or prophylaxis of the specific indication. In general, they are administered in an amount of at least about 10 g/kg body weight and in most cases they will be administered in an amount not in excess of about 8 mg/Kg body weight per day. In most cases, the dosage is from about 10 g/kg to about 1 mg/kg body weight daily, taking into account the routes of administration, symptoms, etc.

The sequences of the present invention are also valuable for chromosome identification. The sequence is specifically targeted to and can hybridize with a particular location on an individual human chromosome. Moreover, there is a current need for identifying particular sites on the chromosome. Few chromosome marking reagents based on actual sequence data (repeat polymorphisms) are presently available for marking chromosomal location. The mapping of DNAs to chromosomes according to the present invention is an important first step in correlating those sequences with genes associated with disease.

Briefly, sequences can be mapped to chromosomes by preparing PCR primers (preferably 15-25 bp) from the cDNA. Computer analysis of the 3′ untranslated region of the gene is used to rapidly select primers that do not span more than one exon in the genomic DNA, thus complicating the amplification process. These primers are then used for PCR screening of somatic cell hybrids containing individual human chromosomes. Only those hybrids containing the human gene corresponding to the primer will yield an amplified fragment.

PCR mapping of somatic cell hybrids is a rapid procedure for assigning a particular DNA to a particular chromosome. Using the present invention with the same oligonucleotide primers, sublocalization can be achieved with panels of fragments from specific chromosomes or pools of large genomic clones in an analogous manner. Other mapping strategies that can similarly be used to map to its chromosome include in situ hybridization, prescreening with labeled flow-sorted chromosomes and preselection by hybridization to construct chromosome specific-cDNA libraries.

Fluorescence in situ hybridization (FISH) of a cDNA clone to a metaphase chromosomal spread can be used to provide a precise chromosomal location in one step. This technique can be used with cDNA as short as 50 or 60 bases. For a review of this technique, see Verma et al., Human Chromosomes: a Manual of Basic Techniques, Pergamon Press, New York (1988).

Once a sequence has been mapped to a precise chromosomal location, the physical position of the sequence on the chromosome can be correlated with genetic map data. Such data are found, for example, in V. McKusick, Mendelian Inheritance in Man (available on line through Johns Hopkins University Welch Medical Library). The relationship between genes and diseases that have been mapped to the same chromosomal region are then identified through linkage analysis (coinheritance of physically adjacent genes).

Next, it is necessary to determine the differences in the cDNA or genomic sequence between affected and unaffected individuals. If a mutation is observed in some or all of the affected individuals but not in any normal individuals, then the mutation is likely to be the causative agent of the disease.

With current resolution of physical mapping and genetic mapping techniques, a cDNA precisely localized to a chromosomal region associated with the disease could be one of between 50 and 500 potential causative genes. (This assumes 1 megabase mapping resolution and one gene per 20 kb).

The polypeptides, their fragments or other derivatives, or analogs thereof, or cells expressing them can be used as an immunogen to produce antibodies thereto. These antibodies can be, for example, polyclonal or monoclonal antibodies. The present invention also includes chimeric, single chain, and humanized antibodies, as well as Fab fragments, or the product of an Fab expression library. Various procedures known in the art may be used for the production of such antibodies and fragments.

Antibodies generated against the polypeptides corresponding to a sequence of the present invention can be obtained by direct injection of the polypeptides into an animal or by administering the polypeptides to an animal, preferably a nonhuman. The antibody so obtained will then bind the polypeptides itself. In this manner, even a sequence encoding only a fragment of the polypeptides can be used to generate antibodies binding the whole native polypeptides. Such antibodies can then be used to isolate the polypeptide from tissue expressing that polypeptide.

For preparation of monoclonal antibodies, any technique which provides antibodies produced by continuous cell line cultures can be used. Examples include the hybridoma technique (Kohler and Milstein, 1975, Nature, 256:495-497), the trioma technique, the human B-cell hybridoma technique (Kozbor et al., 1983, Immunology Today 4:72), and the EBV-hybridoma technique to produce human monoclonal antibodies (Cole, et al., 1985, in Monoclonal Antibodies and Cancer Therapy, Alan R. Liss, Inc., pp. 77-96).

Techniques described for the production of single chain antibodies (U.S. Pat. No. 4,946,778) can be adapted to produce single chain antibodies to immunogenic polypeptide products of this invention. Also, transgenic animals may be used to express humanized antibodies to immunogenic polypeptide products of this invention.

The present invention will be further described with reference to the following examples; however, it is to be understood that the present invention is not limited to such examples. All parts or amounts, unless otherwise specified, are by weight.

In order to facilitate understanding of the following examples certain frequently occurring methods and/or terms will be described.

“Plasmids” are designated by a lower case p preceded and/or followed by capital letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

“Digestion” of DNA refers to catalytic cleavage of the DNA with a restriction enzyme that acts only at certain sequences in the DNA. The various restriction enzymes used herein are commercially available and their reaction conditions, cofactors and other requirements were used as would be known to the ordinarily skilled artisan. For the purpose of isolating DNA fragments for plasmid construction, typically 5 to 50 μg of DNA are digested with 20 to 250 units of enzyme in a larger volume. Appropriate buffers and substrate amounts for particular restriction enzymes are specified by the manufacturer. Incubation times of about 1 hour at 37 C are ordinarily used, but may vary in accordance with the supplier's instructions. After digestion the reaction is electrophoresed directly on a polyacrylamide gel to isolate the desired fragment.

Size separation of the cleaved fragments is performed using 8 percent polyacrylamide gel described by Goeddel, D. et al., Nucleic Acids Res., 8:4057 (1980).

“Oligonucleotides” refers to either a single stranded polydeoxynucleotide or two complementary polydeoxynucleotide strands which may be chemically synthesized. Such synthetic oligonucleotides have no 5′ phosphate and thus will not ligate to another oligonucleotide without adding a phosphate with an ATP in the presence of a kinase. A synthetic oligonucleotide will ligate to a fragment that has not been dephosphorylated.

“Ligation” refers to the process of forming phosphodiester bonds between two double stranded nucleic acid fragments (Maniatis, T., et al., Id., p. 146). Unless otherwise provided, ligation may be accomplished using known buffers and conditions with 10 units of T4 DNA ligase (“ligase”) per 0.5 μg of approximately equimolar amounts of the DNA fragments to be ligated.

Unless otherwise stated, transformation was performed as described in the method of Graham, F. and Van der Eb, A., Virology, 52:456-457 (1973).

### EXAMPLE 1

### Bacterial Expression and Purification of hIAP-1

The DNA sequence encoding hIAP-1, ATCC No. 97141, is initially amplified using PCR oligonucleotide primers corresponding to the 5′ and 3′ end sequences of the processed hIAP-1 gene (minus the signal peptide sequence) and the vector sequences 3′ to the hIAP-1 gene. Additional nucleotides corresponding to hIAP-1 are added to the 5′ and 3′ sequences respectively. The 5′ oligonucleotide primer has the sequence 5′ GATCGGATCCATGAGTACTGAAGAAGCCAG 3′ (SEQ ID NO:3) contains a BamHI restriction enzyme site followed by 20 nucleotides of hIAP-1 coding sequence starting from the presumed terminal amino acid of the processed protein. The 3′ sequence 5′ GACTGGATCCTCTTTAAGAGAGAAATGTACG 3′ (SEQ ID NO:4) contains complementary sequences to a BamHI site. The restriction enzyme sites correspond to the restriction enzyme sites on the bacterial expression vector pQE-9 (Qiagen, Inc. Chatsworth, Calif.). pQE-9 encodes antibiotic resistance (Amp^{r}), a bacterial origin of replication (ori), an WTG-regulatable promoter operator (P/O), a ribosome binding site (RBS), a 6-His tag and restriction enzyme sites. pQE-9 is then digested with BamHI and dephosphorylated. The amplified sequences are ligated into pQE-9 and are inserted in frame with the sequence encoding for the histidine tag and the RBS. The ligation mixture is then used to transform E. coli strain M15/rep 4 (Qiagen, Inc.) by the procedure described in Sambrook, J. et al., Molecular Cloning: A Laboratory Manual, Cold Spring Laboratory Press, (1989). M15/rep4 contains multiple copies of the plasmid pREP4, which expresses the lacI repressor and also confers kanamycin resistance (Kan^{r}). Transformants are identified by their ability to grow on LB plates and ampicillin/kanamycin resistant colonies are selected. Plasmid DNA is isolated and confirmed by restriction analysis. Clones containing the desired constructs are grown overnight (O/N) in liquid culture in LB media supplemented with both Amp (100 ug/ml) and Kan (25 ug/ml). The O/N culture is used to inoculate a large culture at a ratio of 1:100 to 1:250. The cells are grown to an optical density 600 (O.D.⁶⁰⁰) of between 0.4 and 0.6. IPTG (“Isopropyl-B-D-thiogalacto pyranoside”) is then added to a final concentration of 1 mM. IPTG induces by inactivating the lacI repressor, clearing the P/O leading to increased gene expression. Cells are grown an extra 3 to 4 hours. Cells are then harvested by centrifugation. The cell pellet is solubilized in the chaotropic agent 6 Molar Guanidine HCl. After clarification, solubilized hIAP-1 protein is purified from this solution by chromatography on a Nickel-Chelate column under conditions that allow for tight binding by proteins containing the 6-His tag (Hochuli, E. et al., J. Chromatography 411:177-184 (1984)). hIAP-1 (50% pure) is eluted from the column in 6 molar guanidine HCl pH 5.0 and for the purpose of renaturation adjusted to 3 molar guanidine HCl, 100 mM sodium phosphate, 10 mmolar glutathione (reduced) and 2 mmolar glutathione (oxidized). After incubation in this solution for 12 hours the protein is dialyzed to 10 mmolar sodium phosphate.

### EXAMPLE 2

### Cloning and Expression of hIAP-1 Using the Baculovirus Expression System

The DNA sequence encoding the full length hIAP-1 protein, ATCC No. 97141, is amplified using PCR oligonucleotide primers corresponding to the 5′ and 3′ end sequences of the gene:

The 5′ primer has the sequence 5′ GCAGATCTGTAGATCTGGTCACCATGAGTACTG 3′ (SEQ ID NO:5) and contains a BglII restriction enzyme site (in bold) followed by 25 nucleotides resembling an efficient signal for the initiation of translation in eukaryotic cells (Kozak, M., J. Mol. Biol., 196:947-950 (1987) which is just behind the hIAP-1 gene (the initiation codon for translation “ATG” is underlined).

The 3′ primer has the sequence 5′ GCAGATCTTTAAGAGAGAAATGTACG 3′ (SEQ ID NO:6) and contains the cleavage site for the restriction endonuclease BglII and 19 nucleotides complementary to the 3′ non-translated sequence of the hIAP-1 gene. The amplified sequences are isolated from a 1% agarose gel using a commercially available kit (“Geneclean,” BIO 101 Inc., La Jolla, Calif.). The fragment is then digested with the endonuclease BglII and then purified again on a 1% agarose gel. This fragment is designated F2.

The vector pRG1 (modification of pVL941 vector, discussed below) is used for the expression of the hIAP-1 protein using the baculovirus expression system (for review see: Summers, M. D. and Smith, G. E. 1987, A manual of methods for baculovirus vectors and insect cell culture procedures, Texas Agricultural Experimental Station Bulletin NO:1555). This expression vector contains the strong polyhedrin promoter of the Autographa californica nuclear polyhedrosis virus (AcMNPV) followed by the recognition sites for the restriction endonuclease BamHI. The polyadenylation site of the simian virus (SV)40 is used for efficient polyadenylation. For an easy selection of recombinant virus the beta-galactosidase gene from E. coli is inserted in the same orientation as the polyhedrin promoter followed by the polyadenylation signal of the polyhedrin gene. The polyhedrin sequences are flanked at both sides by viral sequences for the cell-mediated homologous recombination of cotransfected wild-type viral DNA. Many other baculovirus vectors could be used in place of pRG1 such as pAc373, pVL941 and pAcIM1 (Luckow, V. A. and Summers, M. D., Virology, 170:31-39).

The plasmid is digested with the restriction enzyme BamHI and dephosphorylated using calf intestinal phosphatase by procedures known in the art. The DNA is then isolated from a 1% agarose gel using the commercially available kit (“Geneclean” BIO 101 Inc., La Jolla, Calif.). This vector DNA is designated V2.

Fragment F2 and the dephosphorylated plasmid V2 are ligated with T4 DNA ligase. E. coli HB101 cells are then transformed and bacteria identified that contained the plasmid (pBac hIAP-1) with the hIAP-1 gene in the correct orientation using PCR. The sequence of the cloned fragment is confirmed by DNA sequencing.

5 μg of the plasmid pBac hIAP-1 is cotransfected with 1.0 μg of a commercially available linearized baculovirus (“BaculoGold baculovirus DNA”, Pharmingen, San Diego, Calif.) using the lipofection method (Felgner et al. Proc. Natl. Acad. Sci. USA, 84:7413-7417 (1987)).

1 μg of BaculoGold virus DNA and 5 μg of the plasmid pBac hIAP-1 are mixed in a sterile well of a microtiter plate containing 50 μl of serum free Grace's medium (Life Technologies Inc., Gaithersburg, Md.). Afterwards 10 μl Lipofectin plus 90 μl Grace's medium are added, mixed and incubated for 15 minutes at room temperature. Then the transfection mixture is added drop-wise to the Sf9 insect cells (ATCC CRL 1711) seeded in a 35 mm tissue culture plate with 1 ml Grace's medium without serum. The plate is rocked back and forth to mix the newly added solution. The plate is then incubated for 5 hours at 27° C. After 5 hours the transfection solution is removed from the plate and 1 ml of Grace's insect medium supplemented with 10% fetal calf serum is added. The plate is put back into an incubator and cultivation continued at 27° C. for four days.

After four days the supernatant is collected and a plaque assay performed similar as described by Summers and Smith (supra). As a modification an agarose gel with “Blue Gal” (Life Technologies Inc., Gaithersburg) is used which allows an easy isolation of blue stained plaques. (A detailed description of a “plaque assay” can also be found in the user's guide for insect cell culture and baculovirology distributed by Life Technologies Inc., Gaithersburg, page 9-10).

Four days after the serial dilution, the virus is added to the cells and blue stained plaques are picked with the tip of an Eppendorf pipette. The agar containing the recombinant viruses is then resuspended in an Eppendorf tube containing 200 μl of Grace's medium. The agar is removed by a brief centrifugation and the supernatant containing the recombinant baculovirus is used to infect Sf9 cells seeded in 35 mm dishes. Four days later the supernatants of these culture dishes are harvested and then stored at 4° C.

Sf9 cells are grown in Grace's medium supplemented with 10% heat-inactivated FBS. The cells are infected with the recombinant baculovirus V-hIAP-1 at a multiplicity of infection (MOI) of 2. Six hours later the medium is removed and replaced with SF900 II medium minus methionine and cysteine (Life Technologies Inc., Gaithersburg, Md.). 42 hours later 5 μCi of ³⁵S-methionine and 5 μCi ³⁵S cysteine (Amersham) are added. The cells are further incubated for 16 hours before they are harvested by centrifugation and the labelled proteins visualized by SDS-PAGE and autoradiography.

### EXAMPLE 3

### Expression of Recombinant hIAP-1 in COS Cells

The expression of plasmid, hIAP-1 HA is derived from a vector pcDNAI/Amp (Invitrogen) containing: 1) SV40 origin of replication, 2) ampicillin resistance gene, 3) E. coli replication origin, 4) CMV promoter followed by a polylinker region, an SV40 intron and polyadenylation site. A DNA fragment encoding the entire hIAP-1 precursor and a HA tag fused in frame to its 3′ end is cloned into the polylinker region of the vector, therefore, the recombinant protein expression is directed under the CMV promoter. The HA tag corresponds to an epitope derived from the influenza hemagglutinin protein as previously described (I. Wilson, H. Niman, R. Heighten, A Cherenson, M. Connolly, and R. Lerner, 1984, Cell 37:767, (1984)). The infusion of HA tag to the target protein allows easy detection of the recombinant protein with an antibody that recognizes the HA epitope.

The plasmid construction strategy is described as follows:

The DNA sequence encoding hIAP-1, ATCC No. 97141, is constructed by PCR using two primers: the 5′ primer 5′ GCAGATCTGCAATGAGTACTGAAGAAGCC 3′ (SEQ ID NO:7) contains a BglII site followed by 21 nucleotides of hIAP-1 coding sequence starting from the initiation codon; the 3′ sequence 5′ GCAGATCTTCAAGCGTAGTCTGGGACGTCGTATGGGTAAGAGAGAAATGTAC GAACAGT 3′ (SEQ ID NO:8) contains complementary sequences to a BglII site, translation stop codon, HA tag and the last 21 nucleotides of the hIAP-1 coding sequence (not including the stop codon). Therefore, the PCR product contains a BglII site, hIAP-1 coding sequence followed by HA tag fused in frame, a translation termination stop codon next to the HA tag, and a BglII site. The PCR amplified DNA fragment is digested with BglII and the vector, pcDNA1/Amp digested with BamHI restriction enzyme are ligated. The ligation mixture is transformed into E. coli strain XL-1-Blue (Stratagene Cloning Systems, La Jolla, Calif.) the transformed culture is plated on ampicillin media plates and resistant colonies are selected. Plasmid DNA is isolated from transformants and examined by restriction analysis for the presence of the correct fragment. For expression of the recombinant hIAP-1, COS cells are transfected with the expression vector by DEAE-DEXTRAN method (J. Sambrook, E. Fritsch, T. Maniatis, Molecular Cloning: A Laboratory Manual, Cold Spring Laboratory Press, (1989)). The expression of the hIAP-1 HA protein is detected by radiolabelling and immunoprecipitation method (E. Harlow, D. Lane, Antibodies: A Laboratory Manual, Cold Spring Harbor Laboratory Press, (1988)). Cells are labelled for 8 hours with ³⁵S-cysteine two days post transfection. Culture media is then collected and cells are lysed with detergent (RIPA buffer (150 mM NaCl, 1% NP-40, 0.1% SDS, 1% NP-40, 0.5% DOC, 50 mM Tris, pH 7.5) (Wilson, I. et al., Id. 37:767 (1984)). Both cell lysate and culture media are precipitated with an HA specific monoclonal antibody. Proteins precipitated are analyzed on 15% SDS-PAGE gels.

### EXAMPLE 4

### Expression of hIAP-1 via Gene Therapy

Fibroblasts are obtained from a subject by skin biopsy. The resulting tissue is placed in tissue-culture medium and separated into small pieces. Small chunks of the tissue are placed on a wet surface of a tissue culture flask, approximately ten pieces are placed in each flask. The flask is turned upside down, closed tight and left at room temperature over night. After 24 hours at room temperature, the flask is inverted and the chunks of tissue remain fixed to the bottom of the flask and fresh media (e.g., Ham's F12 media, with 10% FBS, penicillin and streptomycin, is added. This is then incubated at 37° C. for approximately one week. At this time, fresh media is added and subsequently changed every several days. After an additional two weeks in culture, a monolayer of fibroblasts emerge. The monolayer is trypsinized and scaled into larger flasks.

pMV-7 (Kirschmeier, P. T. et al, DNA, 7:219-25 (1988) flanked by the long terminal repeats of the Moloney murine sarcoma virus, is digested with EcoRI and HindIII and subsequently treated with calf intestinal phosphatase. The linear vector is fractionated on agarose gel and purified, using glass beads.

The cDNA encoding a polypeptide of the present invention is amplified using PCR primers which correspond to the 5′ and 3′ end sequences respectively. The 5′ primer contains an EcoRI site and the 3′ primer contains a HindIII site. Equal quantities of the Moloney murine sarcoma virus linear backbone and the EcoRI and HindII fragment are added together, in the presence of T4 DNA ligase. The resulting mixture is maintained under conditions appropriate for ligation of the two fragments. The ligation mixture is used to transform bacteria HB101, which are then plated onto agar-containing kanamycin for the purpose of confirming that the vector had the gene of interest properly inserted.

The amphotropic pA317 or GP+am12 packaging cells are grown in tissue culture to confluent density in Dulbecco's Modified Eagles Medium (DMEM) with 10% calf serum (CS), penicillin and streptomycin. The MSV vector containing the gene is then added to the media and the packaging cells are transduced with the vector. The packaging cells now produce infectious viral particles containing the gene (the packaging cells are now referred to as producer cells).

Fresh media is added to the transduced producer cells, and subsequently, the media is harvested from a 10 cm plate of confluent producer cells. The spent media, containing the infectious viral particles, is filtered through a millipore filter to remove detached producer cells and this media is then used to infect fibroblast cells. Media is removed from a sub-confluent plate of fibroblasts and quickly replaced with the media from the producer cells. This media is removed and replaced with fresh media. If the titer of virus is high, then virtually all fibroblasts will be infected and no selection is required. If the titer is very low, then it is necessary to use a retroviral vector that has a selectable marker, such as neo or his.

The engineered fibroblasts are then injected into the host, either alone or after having been grown to confluence on cytodex 3 microcarrier beads. The fibroblasts now produce the protein product.

Numerous modifications and variations of the present invention are possible in light of the above teachings and, therefore, within the scope of the appended claims, the invention may be practiced otherwise than as particularly described.

What is claimed is:

1. An isolated antibody or fragment thereof that specifically binds to a protein selected from the group consisting of:
(a) a protein consisting of amino acid residues 1 to 438 of SEQ ID NO:2;
(b) a protein consisting of a portion of SEQ ID NO:2, wherein said portion comprises at least 30 contiguous amino acid residues of SEQ ID NO:2; and
(c) a protein consisting of a portion of SEQ ID NO:2, wherein said portion comprises at least 50 contiguous amino acid residues of SEQ ID NO:2.

2. The antibody or fragment thereof of claim 1 that specifically binds protein (a).

3. The antibody or fragment thereof of claim 1 that specifically binds protein (b).

4. The antibody or fragment thereof of claim 1 that specifically binds protein (c).

5. The antibody or fragment thereof of claim 2 that specifically binds protein (b).

6. The antibody or fragment thereof of claim 2 wherein said protein bound by said antibody or fragment thereof is glycosylated.

7. The antibody or fragment thereof of claim 2 which is a human antibody.

8. The antibody or fragment thereof of claim 2 which is a polyclonal antibody.

9. The antibody or fragment thereof of claim 2 which is selected from the group consisting of:
(a) a chimeric antibody;
(b) a humanized antibody;
(c) a single chain antibody; and
(d) a Fab fragment.

10. The antibody or fragment thereof of claim 2 which is labeled.

11. The antibody or fragment thereof of claim 10 which is labeled with a label selected from the group consisting of:
(a) an enzyme;
(b) a fluorescent label; and
(c) a radioactive label.

12. The antibody or fragment thereof of claim 2 wherein said antibody or fragment thereof specifically binds to said protein in a Western blot.

13. The antibody or fragment thereof of claim 2 wherein said antibody or fragment thereof specifically binds to said protein in an ELISA.

14. An isolated cell that produces the antibody or fragment thereof of claim 2.

15. A hybridoma that produces the antibody or fragment thereof of claim 2.

16. A method of detecting hIAP-1 protein in a biological sample comprising:
(a) contacting the biological sample with the antibody or fragment thereof of claim 2; and
(b) detecting the hIAP-1 protein protein in the biological sample.

17. The method of claim 16 wherein the antibody or fragment thereof is a polyclonal antibody.

18. An isolated antibody or fragment thereof obtained from an animal that has been immunized with a protein selected from the group consisting of:
(a) a protein comprising the amino acid sequence of amino acid residues 1 to 438 of SEQ ID NO:2;
(b) a protein comprising an amino acid sequence of at least 30 contiguous amino acid residues of SEQ ID NO:2; and
(c) a protein comprising an amino acid sequence of at least 50 contiguous amino acid residues of SEQ ID NO:2;
wherein said antibody or fragment thereof specifically binds to said amino acid sequence.

19. The antibody or fragment thereof of claim 18 obtained from an animal immunized with protein (a).

20. The antibody or fragment thereof of claim 18 obtained from an animal immunized with protein (b).

21. The antibody or fragment thereof of claim 18 obtained from an animal immunized with protein (c).

22. The antibody or fragment thereof of claim 18 which is a monoclonal antibody.

23. The antibody or fragment thereof of claim 18 which is selected from the group consisting of:
(a) a chimeric antibody;
(b) a polyclonal antibody;
(c) a humanized antibody;
(d) a single chain antibody; and
(e) a Fab fragment.

24. An isolated monoclonal antibody or fragment thereof that specifically binds to a protein selected from the group consisting of:
(a) a protein consisting of amino acid residues 1 to 438 of SEQ ID NO:2;
(b) a protein consisting of a portion of SEQ ID NO:2, wherein said portion comprises at least 30 contiguous amino acid residues of SEQ ID NO:2; and
(c) a protein consisting of a portion of SEQ ID NO:2, wherein said portion comprises at least 50 contiguous amino acid residues of SEQ ID NO:2.

25. The antibody or fragment thereof of claim 24 that specifically binds protein (a).

26. The antibody or fragment thereof of claim 24 that specifically binds protein (b).

27. The antibody or fragment thereof of claim 24 that specifically binds protein (c).

28. The antibody or fragment thereof of claim 25 that specifically binds protein (b).

29. The antibody or fragment thereof of claim 25 wherein said protein bound by said antibody or fragment thereof is glycosylated.

30. The antibody or fragment thereof of claim 25 which is a human antibody.

31. The antibody or fragment thereof of claim 25 which is selected from the group consisting of:
(a) a chimeric antibody;
(b) a humanized antibody;
(c) a single chain antibody; and
(d) a Fab fragment.

32. The antibody or fragment thereof of claim 25 which is labeled.

33. The antibody or fragment thereof of claim 32 which is labeled with a label selected from the group consisting of:
(a) an enzyme;
(b) a fluorescent label; and
(c) a radioactive label.

34. The antibody or fragment thereof of claim 25 wherein said antibody or fragment thereof specifically binds to said protein in a Western blot.

35. The antibody or fragment thereof of claim 25 wherein said antibody or fragment thereof specifically binds to said protein in an ELISA.

36. An isolated cell that produces the antibody or fragment thereof of claim 25.

37. A hybridoma that produces the antibody or fragment thereof of claim 25.

38. A method of detecting hIAP-1 protein in a biological sample comprising:
(a) contacting the biological sample with the antibody or fragment thereof of claim 25; and
(b) detecting the hIAP-1 protein in the biological sample.

39. An isolated antibody or fragment thereof that specifically binds to a protein selected from the group consisting of:
(a) a protein consisting of the full-length polypeptide encoded by the cDNA contained in ATCC Deposit Number 97141;
(b) a protein consisting of the mature form of the polypeptide encoded by the cDNA contained in ATCC Deposit Number 97141;
(c) a protein consisting of a portion of the polypeptide encoded by the cDNA contained in ATCC Deposit Number 97141, wherein said portion comprises at least 30 contiguous amino acid residues of the polypeptide encoded by the cDNA contained in ATCC Deposit Number 97141; and
(d) a protein consisting of a portion of the polypeptide encoded by the cDNA contained in ATCC Deposit Number 97141, wherein said portion comprises at least 50 contiguous amino acid residues of the polypeptide encoded by the cDNA contained in ATCC Deposit Number 97141.

40. The antibody or fragment thereof of claim 39 that specifically binds protein (a).

41. The antibody or fragment thereof of claim 39 that specifically binds protein (b).

42. The antibody or fragment thereof of claim 39 that specifically binds protein (c).

43. The antibody or fragment thereof of claim 39 that specifically binds protein (d).

44. The antibody or fragment thereof of claim 40 that specifically binds protein (b).

45. The antibody or fragment thereof of claim 40 wherein said protein bound by said antibody or fragment thereof is glycosylated.

46. The antibody or fragment thereof of claim 40 which is a human antibody.

47. The antibody or fragment thereof of claim 40 which is a polyclonal antibody.

48. The antibody or fragment thereof of claim 40 which is selected from the group consisting of:
(a) a chimeric antibody;
(b) a humanized antibody;
(c) a single chain antibody; and
(d) a Fab fragment.

49. The antibody or fragment thereof of claim 40 which is labeled.

50. The antibody or fragment thereof of claim 49 which is labeled with a label selected from the group consisting of:
(a) an enzyme;
(b) a fluorescent label; and
(c) a radioactive label.

51. The antibody or fragment thereof of claim 40 wherein said antibody or fragment thereof specifically binds to said protein in a Western blot.

52. The antibody or fragment thereof of claim 40 wherein said antibody or fragment thereof specifically binds to said protein in an ELISA.

53. An isolated cell that produces the antibody or fragment thereof of claim 40.

54. A hybridoma that produces the antibody or fragment thereof of claim 40.

55. A method of detecting hIAP-1 protein in a biological sample comprising:
(a) contacting the biological sample with the antibody or fragment thereof of claim 40; and
(b) detecting the hIAP-1 protein in the biological sample.

56. The method of claim 55 wherein the antibody or fragment thereof is a polyclonal antibody.

57. An isolated antibody or fragment thereof obtained from an animal that has been immunized with a protein selected from the group consisting of:
(a) a protein comprising the amino acid sequence of the full-length polypeptide encoded by the cDNA contained in ATCC Deposit Number 97141;
(b) a protein comprising the amino acid sequence of the mature form of the polypeptide encoded by the cDNA contained in ATCC Deposit Number 97141;
(c) a protein comprising an amino acid sequence of at least 30 contiguous amino acid residues of the polypeptide encoded by the cDNA contained in ATCC Deposit Number 97141; and
(d) a protein comprising an amino acid sequence of at least 50 contiguous amino acid residues the polypeptide encoded by the cDNA contained in ATCC Deposit Number 97141;
wherein said antibody or fragment thereof specifically binds to said amino acid sequence.

58. The antibody or fragment thereof of claim 57 obtained from an animal immunized with protein (a).

59. The antibody or fragment thereof of claim 57 obtained from an animal immunized with protein (b).

60. The antibody or fragment thereof of claim 57 obtained from an animal immunized with protein (c).

61. The antibody or fragment thereof of claim 57 obtained from an animal immunized with protein (d).

62. The antibody or fragment thereof of claim 57 which is a monoclonal antibody.

63. The antibody or fragment thereof of claim 57 which is selected from the group consisting of:
(a) a chimeric antibody;
(b) a polyclonal antibody;
(c) a humanized antibody;
(d) a single chain antibody; and
(e) a Fab fragment.

64. An isolated monoclonal antibody or fragment thereof that specifically binds to a protein selected from the group consisting of:
(a) a protein consisting of the full-length polypeptide encoded by the cDNA contained in ATCC Deposit Number 97141;
(b) a protein consisting of the mature form of the polypeptide encoded by the cDNA contained in ATCC Deposit Number 97141;
(c) a protein consisting of a portion of the polypeptide encoded by the cDNA contained in ATCC Deposit Number 97141, wherein said portion comprises at least 30 contiguous amino acid residues of the polypeptide encoded by the cDNA contained in ATCC Deposit Number 97141; and
(d) a protein consisting of a portion of the polypeptide encoded by the cDNA contained in ATCC Deposit Number 97141, wherein said portion comprises at least 50 contiguous amino acid residues of the polypeptide encoded by the cDNA contained in ATCC Deposit Number 97141.

65. The antibody or fragment thereof of claim 64 that specifically binds protein (a).

66. The antibody or fragment thereof of claim 64 that specifically binds protein (b).

67. The antibody or fragment thereof of claim 64 that specifically binds protein (c).

68. The antibody or fragment thereof of claim 64 that specifically binds protein (d).

69. The antibody or fragment thereof of claim 65 that specifically binds protein (b).

70. The antibody or fragment thereof of claim 65 wherein said protein bound by said antibody or fragment thereof is glycosylated.

71. The antibody or fragment thereof of claim 65 which is a human antibody.

72. The antibody or fragment thereof of claim 65 which is selected from the group consisting of:
(a) a chimeric antibody;
(b) a humanized antibody;
(c) a single chain antibody; and
(d) a Fab fragment.

73. The antibody or fragment thereof of claim 65 which is labeled.

74. The antibody or fragment thereof of claim 73 which is labeled with a label selected from the group consisting of:
(a) an enzyme;
(b) a fluorescent label; and
(c) a radioactive label.

75. The antibody or fragment thereof of claim 65 wherein said antibody or fragment thereof specifically binds to said protein in a Western blot.

76. The antibody or fragment thereof of claim 65 wherein said antibody or fragment thereof specifically binds to said protein in an ELISA.

77. An isolated cell that produces the antibody or fragment thereof of claim 65.

78. A hybridoma that produces the antibody or fragment thereof of claim 65.

79. A method of detecting hIAP-1 protein in a biological sample comprising:
(a) contacting the biological sample with the antibody or fragment thereof of claim 65; and
(b) detecting the hIAP-1 protein in the biological sample.

80. An isolated antibody or fragment thereof that specifically binds a hIAP-1 protein expressed in a cell wherein said hIAP-1 protein is encoded by a polynucleotide encoding amino acids 1 to 438 of SEQ ID NO:2 operably associated with a regulatory sequence that controls the expression of said polynucleotide.

81. The antibody or fragment thereof of claim 80 which is a monoclonal antibody.

82. The antibody or fragment thereof of claim 80 which is a human antibody.

83. The antibody or fragment thereof of claim 80 which is selected from the group consisting of:
(a) a chimeric antibody;
(b) a polyclonal antibody;
(c) a humanized antibody;
(d) a single chain antibody; and
(e) a Fab fragment.

84. The antibody or fragment thereof of claim 80 wherein said antibody or fragment thereof specifically binds to said protein in a Western blot.

85. The antibody or fragment thereof of claim 80 wherein said antibody or fragment thereof specifically binds to said protein in an ELISA.


SEQUENCE LISTING (10 sequences)

SEQ ID NO: 1 - DNA, 1435 bp, Homo sapiens
  CDS (10)..(1326)
agttatgcaatgagtactgaagaagccagatttcttacctaccatatgtggccattaacctttttgtcaccatcagaattggcaagagctggtttttattatataggacctggagatagggtagcctgctttgcctgtggtgggaagctcagtaactgggaaccaaaggatgatgctatgtcagaacaccggaggcattttcccaactgtccatttttggaaaattctctagaaactctgaggtttagcatttcaaatctgagcatgcagacacatgcagctcgaatgagaacatttatgtactggccatctagtgttccagttcagcctgagcagcttgcaagtgctggtttttattatgtgggtcgcaatgatgatgtcaaatgcttttgttgtgatggtggcttgaggtgttgggaatctggagatgatccatgggtagaacatgccaagtggtttccaaggtgtgagttcttgatacgaatgaaaggccaagagtttgttgatgagattcaaggtagatatcctcatcttcttgaacagctgttgtcaacttcagataccactggagaagaaaatgctgacccaccaattattcattttggacctggagaaagttcttcagaagatgctgtcatgatgaatacacctgtggttaaatctgccttggaaatgggctttaatagagacctggtgaaacaaacagttcaaagtaaaatcctgacaactggagagaactataaaacagttaatgatattgtgtcagcacttcttaatgctgaagatgaaaaaagagaagaggagaaggaaaaacaagctgaagaaatggcatcagatgatttgtcattaattcggaagaacagaatggctctctttcaacaattgacatgtgtgcttcctatcctggataatcttttaaaggccaatgtaattaataaacaggaacatgatattattaaacaaaaaacacagatacctttacaagcgagagaactgattgataccattttggttaaaggaaatgctgcggccaacatcttcaaaaactgtctaaaagaaattgactctacattgtataagaacttatttgtggataagaatatgaagtatattccaacagaagatgtttcaggtctgtcactggaagaacaattgaggaggttgcaagaagaacgaacttgtaaagtgtgtatggacaaagaagtttctgttgtatttattccttgtggtcatctggtagtatgccaggaatgtgccccttctctaagaaaatgccctatttgcaggggtataatcaagggtactgttcgtacatttctctcttaaagaaaaatagtctatattttaacctgcataaaaaggtctttaaaatattgttgaacacttgaagccatctaaagtaaaaagggaattatgagtttttcaattagtaaca

SEQ ID NO: 2 - protein, 438 aa, Homo sapiens
MSTEEARFLTYHMWPLTFLSPSELARAGFYYIGPGDRVACFACGGKLSNWEPKDDAMSEHRRHFPNCPFLENSLETLRFSISNLSMQTHAARMRTFMYWPSSVPVQPEQLASAGFYYVGRNDDVKCFCCDGGLRCWESGDDPWVEHAKWFPRCEFLIRMKGQEFVDEIQGRYPHLLEQLLSTSDTTGEENADPPIIHFGPGESSSEDAVMMNTPVVKSALEMGFNRDLVKQTVQSKILTTGENYKTVNDIVSALLNAEDEKREEEKEKQAEEMASDDLSLIRKNRMALFQQLTCVLPILDNLLKANVINKQEHDIIKQKTQIPLQARELIDTILVKGNAAANIFKNCLKEIDSTLYKNLFVDKNMKYIPTEDVSGLSLEEQLRRLQEERTCKVCMDKEVSVVFIPCGHLVVCQECAPSLRKCPICRGIIKGTVRTFLS

SEQ ID NO: 3 - DNA, 30 bp, Artificial
  Primer
gatcggatccatgagtactgaagaagccag

SEQ ID NO: 4 - DNA, 31 bp, Artificial
  Primer
gactggatcctctttaagagagaaatgtacg

SEQ ID NO: 5 - DNA, 33 bp, Artificial
  Primer
gcagatctgtagatctggtcaccatgagtactg

SEQ ID NO: 6 - DNA, 26 bp, Artificial
  Primer
gcagatctttaagagagaaatgtacg

SEQ ID NO: 7 - DNA, 29 bp, Artificial
  Primer
gcagatctgcaatgagtactgaagaagcc

SEQ ID NO: 8 - DNA, 59 bp, Artificial
  Primer
gcagatcttcaagcgtagtctgggacgtcgtatgggtaagagagaaatgtacgaacagt

SEQ ID NO: 9 - protein, 275 aa, Cydia pomonella granulovirus
MSDLRLEEVRLNTFEKWPVSFLSPETMAKNGFYYLGRSDEVRCAFCKVEIMRWKEGEDPAADHKKWAPQCPFVKGIDVCGSIVTTNNIQNTTTHDTIIGPAHPKYAHEAARVKSFHNWPRCMKQRPEQMADAGFFYTGYGDNTKCFYCDGGLKDWEPEDVPWEQHVRWFDRCAYVQLVKGRDYVQKVITEACVLPGENTTVSTAAPVSEPIPETKIEKEPQVEDSKLCKICYVEECIVCFVPCGHVVACAKCALSVDKCPMCRKIVTSVLKVYFS

SEQ ID NO: 10 - protein, 268 aa, Orgyia pseudotsugata nuclear polyhedrosis virus
MSSRAIGAPQEGADMKNKAARLGTYTNWPVQFLEPSRMAASGFYYLGRGDEVRCAFCKVEITNWVRGDDPETDHKRWAPQCPFVRNNAHDTPHDRAPPARSAAAHPQYATEAARLRTFAEWPRGLKQRPEELAEAGFFYTGQGDKTRCFCCDGGLKDWEPDDAPWQQHARWYDRCEYVLLVKGRDFVQRVMTEACVVRDADNEPHIERPAVEAEVADDRLCKICLGAEKTVCFVPCGHVVACGKCAAGVTTCPVCRGQLDKAVRMYQV